(19) United States
(12) Patent Application Publication
(10) Pub. No.: US 2009/0025733 A1
(43) Pub. Date: Jan. 29, 2009

(54) Shermo

(76) Inventor: Regina L. Lytle, Fredericksburg, VA (US)

Correspondence Address: Regina Lytle, 5501 Merchants View Square # 159, Haymarket, VA, 20169, US

(21) Appl. No.: 11/804,321

(22) Filed: Jul. 23, 2007

(51) Int. Cl. A61F 13/00; A41D 13/00

(52) U.S. Cl. 128/846; 2/455

(57) ABSTRACT

The present invention (shermo) is a thin protective lining (1 mm in thickness, 6.5 inches in length and 2.5 inches in width) (FIG. 1), which provides comfort potential and simultaneously lessens the possibility of contracting or transferring diseases when trying on below-the-waist garments with or without wearing undergarments. The shermo has a smooth paper-like topside (a) and an adhesive underside (b) (as shown in FIG. 2). The shermo will be retrieved from a wall-mounted dispenser, and the adhesive underside is placed in the crouch of the garment and the topside is facing the wearer's crouch. Once the wearer has used the shermo it will be discarded.

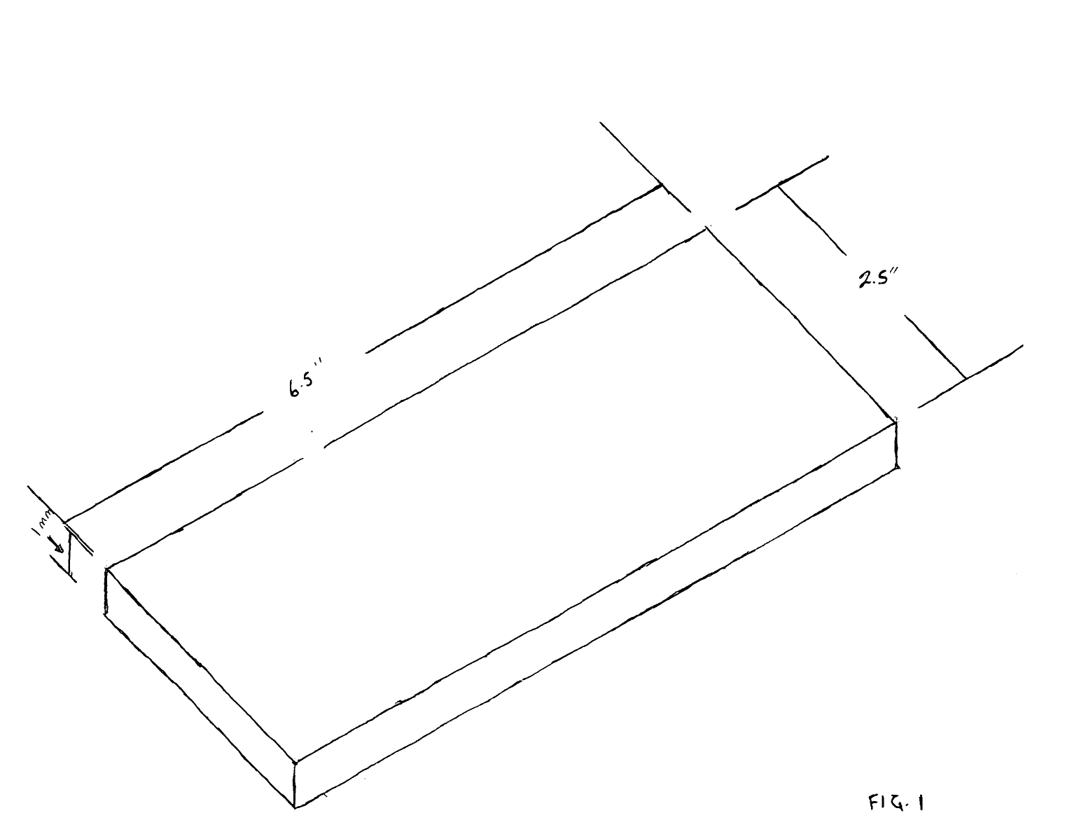

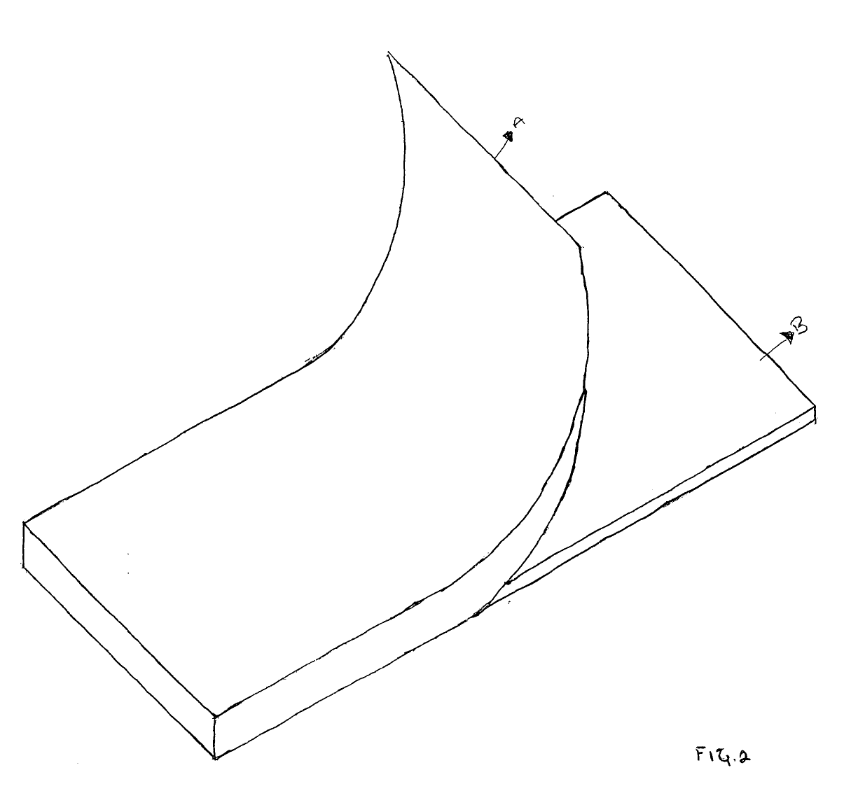

To be used as a sheild liner or of any capacity thereof. In pants, underware, bathing suits, shorts, et. al for men, women and children of all sizes and ages—when trying on clothes.

I claim:

1. A disposable thin, protective article to be fitted in the crotch of the garment and the said cotton topside is facing the wearer's crotch and said adhesive back-sheet is facing the wearer's garment. Said article is used when trying on garments with or without wearing undergarments. Said article simultaneously lessens the possibility of contracting diseases and or bacterial infections.
